(19) United States
(12) Patent Application Publication
(10) Pub. No.: US 2015/0011817 A1
(43) Pub. Date: Jan. 8, 2015

(54) System and Method for Delivering an Ultra-High Dose of Radiation Therapy

(71) Applicant: Yuxin Feng, Tuscaloosa, AL (US)

(72) Inventor: Yuxin Feng, Tuscaloosa, AL (US)

(21) Appl. No.: 14/323,087

(22) Filed: Jul. 3, 2014

Related U.S. Application Data
(60) Provisional application No. 61/842,542, filed on Jul. 3, 2013.

(51) Int. Cl. A61N 5/10

(52) U.S. Cl. CPC A61N 5/1031; A61N 5/1077
USPC 600/1

(57) ABSTRACT

Ultra high dose rate approach was proposed to irradiate to a moving target in radiation therapy in which the prescribed radiation dose was delivered within such a short time period that the displacement of the target could be ignored during dose delivering. The advantages of the approach were evaluated based on normal tissue sparing, flexibility of accuracy of targeting, and time saving in clinical treatment. A system and method of generating of ultra high dose rate combines and utilizes both a linear accelerator and a storage ring.

The current application claims a priority to the U.S. Provisional Patent application Ser. No. 61/842,542 filed on Jul. 3, 2013.

### FIELD OF THE INVENTION

The present invention generally relates to a system and method for radiation therapy, utilizing rapid delivery of radiation in order to minimize damage to healthy tissue and allow for higher doses to be directed towards affected tumor.

### BACKGROUND OF THE INVENTION

In radiation therapy, both cancer cells and normal cells are killed. The clinically acceptable outcome of tumor control (TC) and normal tissue complicity (NTC) can be achieved by catering a radiation dose to target a tumor and to spare normal tissue. When the tumor is moving due to breathing or brow movement, it is challenging to administer the catered radiation dose to the tumor and to spare the normal tissue. Improper technique may lead to applying an insufficient dose to the tumor and to applying an overdose to the normal tissue. Three major approaches have been developed in order to address tumor motion issue in radiation therapy.

The first approach is to add a broader margin to the contour of a target in an attempt to account for displacement of the target due to the patient's physical movements. The broader margin includes a larger volume of normal tissue surrounding tumor in the target and increases the probability of NTC. In order to increase the probability of TC and to decrease the probability of NTC, a hyper-fractionation radiation treatment has been applied to prevent the repair of tumor cells and to allow for the repair of normal cells. It increases length of treatment courses.

The second approach is real-time adaptive radiation therapy, wherein variations in the target are compensated with beam modification components or patients' setup adjustment. Those variations in position, shaper, speed, and etc. are derived from tracking with imaging modalities during delivery of the radiation dose, which usually requires a smaller margin than that in the first approach. The optimized dose distribution is dependent on the position and shape of the target and the organs in risk to exposure. The real-time adaptive approach has been an on-going area of research in order to solve problems with distributing an optimized dose exactly as planned. This is because there is a time delay for adjusting the beam modification components or adjusting the patients' setup when a variation occurs within the target. Thus, the application of the adaptive approach has been limited in clinical practice. Modifying the beam modification components to track the target's motion without dose calculation may have less of a time delay but it not always enough to distribute an optimized dose.

One example of real-time adaptive radiation therapy is respiratory gating, in which the delivering of radiation dose is gated to the durations when the target is in a selected region, according to imaging tracking or monitoring of surrogate. The selected region was used as the target for treatment planning. As the margin was reduced, the duration region becomes shorter. The prescribed dose may have to be separately delivered into multiple shorter sections. It may lead to longer delivering time and larger variations in the position and shaper of the target.

The third approach is emission gated radiation therapy (EGRT) in which the cancer cells were attached (labeled, marked) with radiation pharmaceutical agents, such as 18F FDG, and radiation dose was delivered at a direction along the line of a instant detected pair event of an annihilation of a positron. However, it is still an unresolved challenge to delivering optimized dose distribution to cover a planned target volume and sparing normal tissue surrounding the target.

The uncertainty in treating moving target in radiation therapy may be greatly reduced by delivering the radiation dose in such a short time period when the displacement of tumor and variations in patients' setup could be ignored with an ultra-high dose rate. The advantages of this approach are able to increase accuracy of delivering and sparing more normal tissue by reducing the margin that added to target to encounter displacement of tumor.

In the following sections, we will evaluation the advantages of accuracy improvement, margin reduction, and time saving with delivering the radiation dose with an ultra-high dose rate in section I, the strategies of image guiding delivering in section II, a description of the innovation of ultra-high dose rate system in section III.

### I. Advantages

### Accuracy Improvement

To reduce the variation between delivered dose and optimized dose generated from treatment planning system, the dose should be delivered at the same condition as that used in planning. Combining comprehensive imaging tracking techniques with the ultra-fast dose delivering technique, the planned dose could be delivered within such a short time period with the negligible variation in the position and shaper of target between delivering and planning. The modulation of dose distribution could be achieved with static or dynamic compensator. There is no need of adaptive approach.

### Sparing Normal Tissues

In general, the movement of target was caused by periodical movements of breathing at the time scale of ˜5 second and cardiac motion at ˜1 second, and almost random movement of blows. With the ultra-fast dose deliver, for an example delivering dose within ˜0.01 second, the displacement of target was ignored during delivering. In this case the margin that encountered movement of target could be significantly reduced. At conventional dose rate the margin added to gross tumor volume (GTV) to form internal target volume (ITV) was based on the maximum displacement of periodical movement of GTV at the time scale of breathing for an example because the duration of delivering was usually longer than the period of periodical movements.

In the case of ultra-fast dose delivering, the displacement of GTV (d) could be determined by the multiplication of duration of delivering (τ) and speed of GTV (υ): In general, the velocity of GTV was about a one centimeter per second, the margin add GTV to create ITV could be ˜0.01 millimeter for ˜0.01 second dose delivering. Even taking into account the time delay ˜0.03 second of the verification of delivering conditions, the margin could be reasonably set to 0.5 millimeter that was significantly smaller than that margin currently applied on GTV to form ITV.

For an example, the volumes of ITV were significantly increased while ITV was generated by merging GTV contoured in each phase of a breathing cycle. To illustrate the increasing volume of ITV from the clinical target volume (CTV), the volumes of CTV and ITV were extracted from four-dimensional computed tomography (4DCT) data of 20 patients with lung cancer and treated with stereotactic body radiation therapy (SBRT).

Furthermore, the margin added to ITV to generate PVT (Planning target volume) could be reduced also. The margin was account for setup uncertainty of patient setup due to uncertainty of imaging and intra-fraction variation of patient's position in imaging guided radiation therapy (IGRT). The variation of intra-fraction patient positioning could be eliminated, if the patient position was verified to be the same as that of planning when the planned dose was delivering. The verification could be conducted by taking orthogonal images right before dose delivering. The margin reduction could improve normal tissue sparing, especially for large tumors and pediatric patients. For a tumor with demission (r) and adding a margin (dr) to form a target, the increased volume of target (V) could be approximately represented as: V∝r2dr.

### Shortening the Treatment Time

The radiation therapy with ultra-fast dose delivering was able to short treatment time in two ways: 1) delivering time; 2) gating time. Firstly, it is obvious that the ultra-high dose rate allow the prescribed dose delivered in a much shorter time period than that in conventional radiation therapy with dose rate of ˜1000 Mu/min. However, the ultra-high dose rate prevented the modulation of radiation intensity with moving parts, such as multi-leaf collimation system applied in most intensity modulated radiation therapy (IMRT). The compensator can be an alternative of multiple leave system and allows the ultra-fast dose delivering system to accomplish IMRT. A compensator with the capability of real time justification can used also to eliminate the time to replace the compensators manually, for an example a liquid metal filling system.

The ultra-fast dose delivering also allows reducing the treatment time significantly in gated radiation treatment. The gating technique has been used to treatment a moving target by delivering dose at a selected period when the tumor was at an expected location as that used in treatment planning system. In order to reduce the margin that accounts the displacement of tumor, the delivering period was shortened. The dose was usually delivered in many fractions that required longer treatment time because the low dose rate. The ultra-fast delivering technique allows narrowing the gating window to achieve a higher precision in gating without increasing the time for dose delivering and the dose could be delivered in one of the period when the target was at the same position as that used in treatment planning.

Furthermore, the ultra-fast delivering technique makes some motion management approaches and delivering strategies practically feasible for more cases, such as breath holding and delivering dose at optimized target positions, and etc.

### II. The Strategies of Image Guiding with Ultra-Fast Dose Delivering

The process of imaging gating ultra-fast dose delivering combines target tracking, verification, and dose delivering. There is no requirement of adaptive process, such as changing treatment plan according to the variation derived from imaging registration of the tracked target. With the ultra-fast delivering technique, the dose could be delivered in such a short time period during which variation of the tumor position and patient setup are negligible between what had been used in planning and that at delivering. Furthermore, the tumor position could be selected based on 4DCT when it allows optimizing of tumor coverage and normal tissue sparing in treatment planning.

To catch the delivering condition as specified in treatment planning, orthogonal KV fluoroscopy could be applied. The images were matched with digital reconstructed radiographic (DRR) images in real time. The DRR images were generated from CT data site used in treatment planning system. In these strategies, there was no imaging registration required to derive information for adaptive planning that was time-consuming due to intensive computation. The ultra-fast delivering approach greatly simplified the treatment. The scheme of the treatment could be represented in FIG. 6.

### BRIEF DESCRIPTION OF THE DRAWINGS

FIG. 1 is a diagram of the present invention showing a particle beam from a linear accelerator being injected into a storage ring.

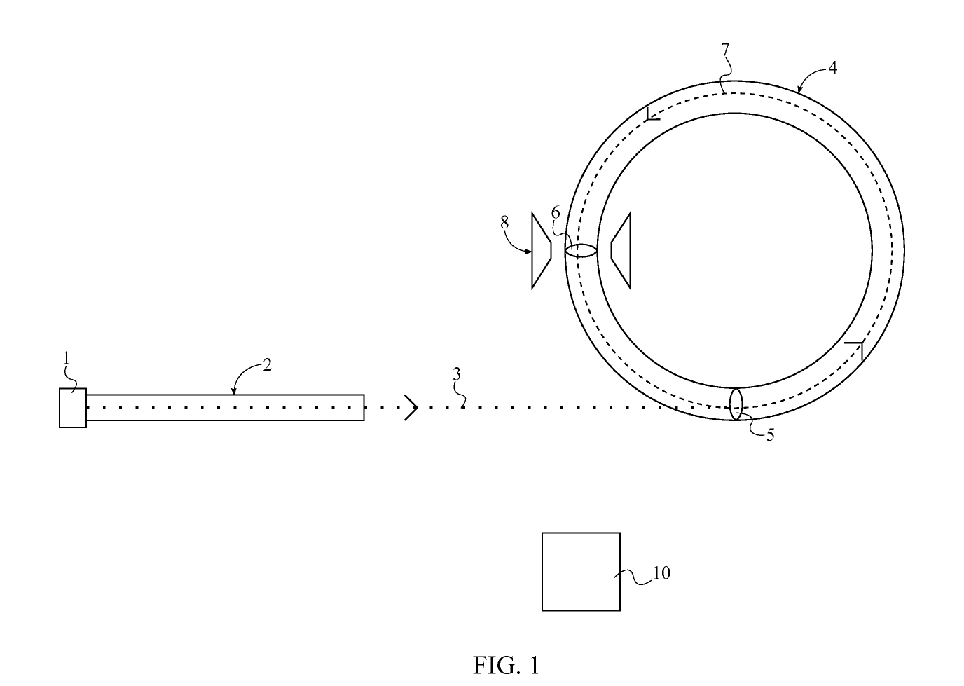

FIG. 2 is a diagram of the present invention showing cycling the particle beam within the storage ring.

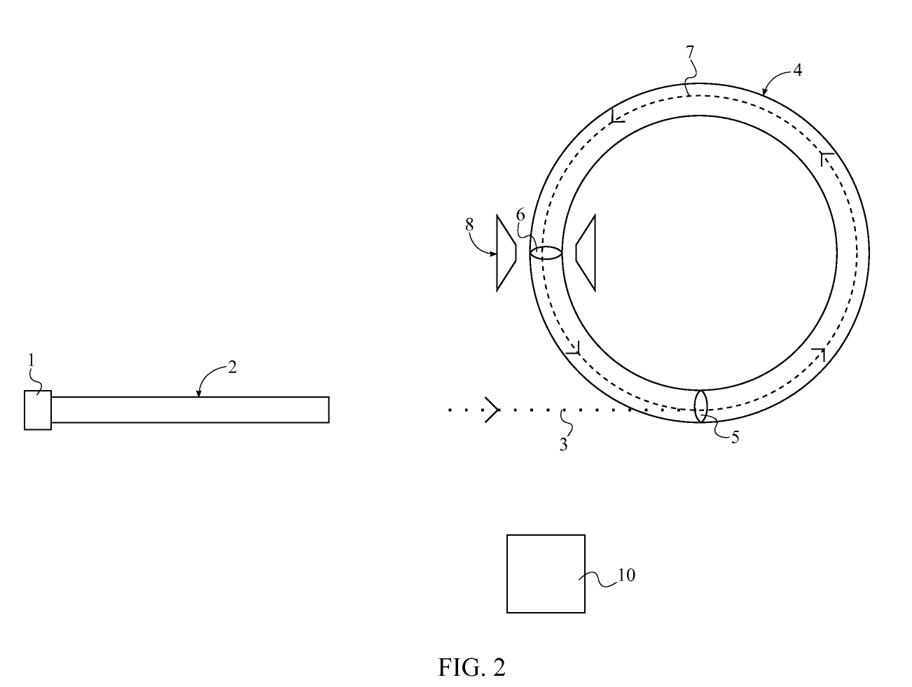

FIG. 3 is a diagram of the present invention showing a switcher being used to redirect the particle beam towards a target delivery system.

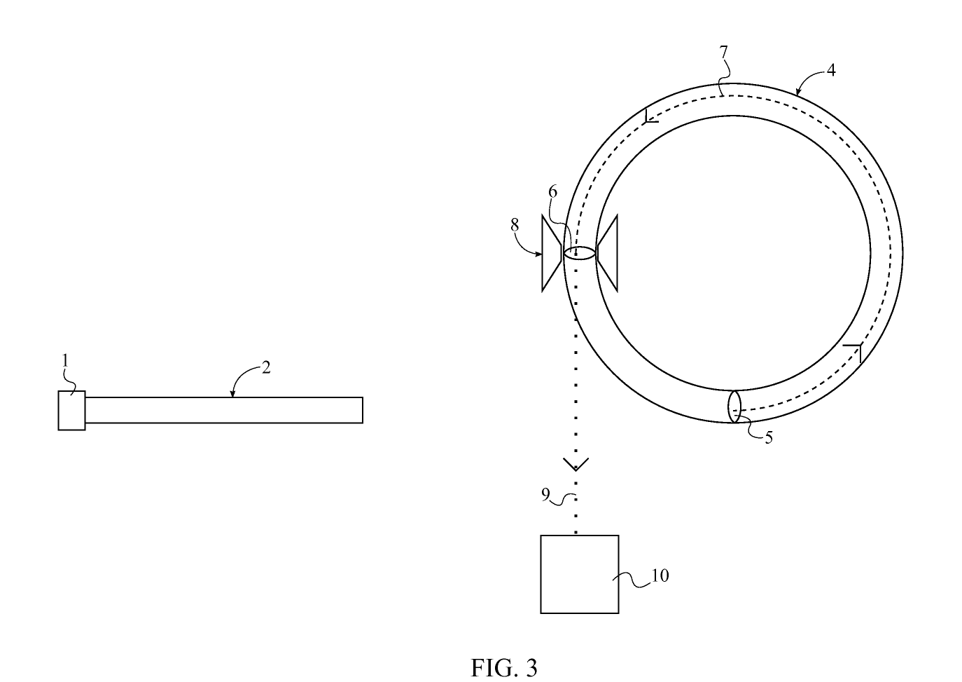

FIG. 4 is a chart outlining the general process of the present invention.

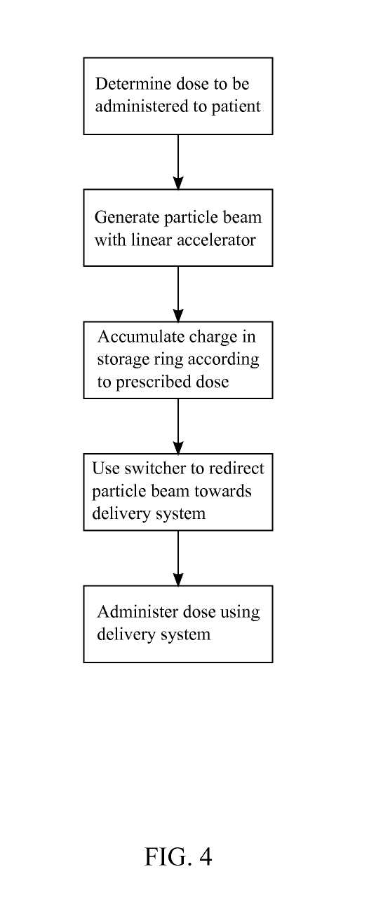

FIG. 5 is a chart outlining adjustments which can be made to adjust the lifetime of contained particles in the storage ring.

FIG. 6 is a chart outlining the process of image guiding.

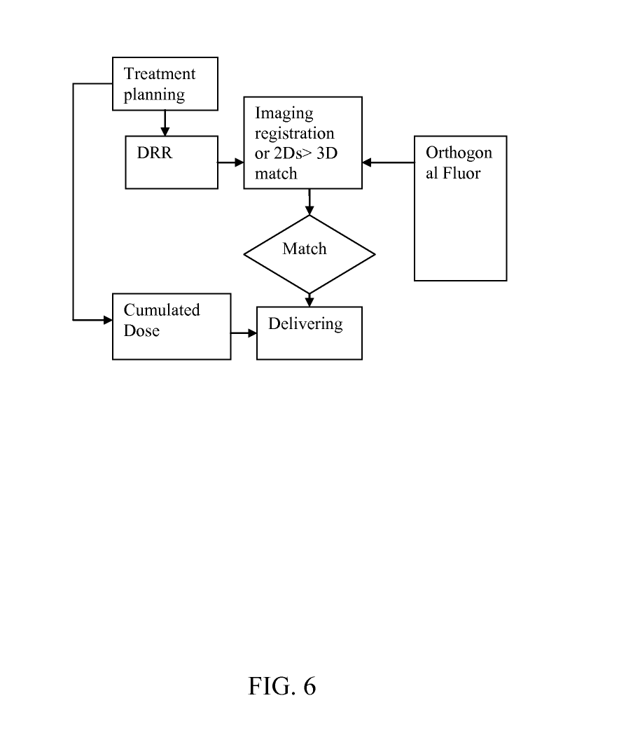

### DETAILED DESCRIPTION OF THE INVENTION

All illustrations of the drawings are for the purpose of describing selected versions of the present invention and are not intended to limit the scope of the present invention.

The present invention is a system and a method for delivering an ultra-high dose rate in radiation therapy. The system for the present invention comprises a particle generator 1, a linear accelerator 2, a storage ring 4, a switcher 8, and a delivery system 10. The particle generator 1 is used to produce a specific kind of atomic or subatomic particle that will later create ionizing radiation with the ultra-high dose rate. The linear accelerator 2 is used to accelerate these particles so that these particles enter the storage ring 4 with a large amount of kinetic energy. The present invention stores the high-energy particles by circulating them about the storage ring 4, which allows the high-energy particles to be accumulated and readily available as a means to deliver the ultra-high dose to a treatment target. Typically, the treatment target is tumor cells that are surrounded by normal tissue and is moving due to natural bodily functions such as breathing. The storage ring 4 holds the high-energy particles until the treatment target is in the optimal position to administer the ultra-high dose rate to the tumor and to spare the normal tissue. The switcher 8 allows the storage ring 4 to release those particles towards the delivery system 10. Moreover, the delivery system 10 is used to modify the beam of high-energy particles into an appropriate form of ionizing radiation that is administered to the treatment target.

The general configuration of the components allows the present invention to efficiently and effectively generate, accelerate, store, and optimally administer high-energy particles with an ultra-high radiation dose rate to the treatment target. The particle generator 1 is oriented into the linear accelerator 2 so that the linear accelerator 2 can immediately accelerate the particles once they are produced by the particle generator 1. The present invention is designed to allow these particles to travel along an acceleration path 3 for the linear accelerator 2 and to travel around an annular storage path 7 for the storage ring 4. The linear accelerator 2 and the storage ring 4 are configured in such a way that the acceleration path 3 is tangent to the annular storage path 7, which allows the particles to seamlessly travel from the linear accelerator 2 into the storage ring 4. Similarly, the present invention is designed to allow these particles to travel along a release path 9 for the storage ring 4. The release path 9 is oriented towards the delivery system 10 so that those particles travel towards the delivery system 10 once they are released from the storage ring 4. The delivery system 10 and the storage ring 4 are also configured in such a way that the release path 9 is tangent to the annular storage path 7, which allows the particles to seamlessly travel from the storage ring 4 towards the delivery system 10. The acceleration path 3, the annular storage path 7, and the release path 9 are coplanar to each other so that the present invention is able to guide the high-energy particles from the linear accelerator 2, through the storage ring 4, and to the delivery system 10 with minimal effort. In the preferred embodiment of the present invention, the acceleration path 3 and the release path 9 are directional paths and are oriented at an angle of 270 degrees.

The storage ring 4 is a critical component to the present invention because the storage ring 4 allows the present invention is able to hold high-energy particles in an orbit around the annular storage path 7 until certain amount of high-energy particles are accumulated and the treatment target is in the optimal position to receive the dose at the ultra-high radiation dose rate. The storage ring 4 comprises an input port 5 and an output port 6. The input port 5 allows particles to enter the storage ring 4, and, consequently, the acceleration path 3 is coincident with the input port 5. Likewise, the output port 6 allows the particles to exit the storage ring 4, and, thus, the release path 9 is coincident with the output port 6. The switcher 8 is also operatively coupled to the output port 6 so that the particles within the storage ring 4 are immediately released through the output port 6 once the switcher 8 is activated. Moreover, the linear accelerator 2 is offset from the storage ring 4 so that the release path 9 does not intersect into the linear accelerator 2. This would prevent the high-energy particles from properly exiting the storage ring 4. Similarly, the delivery system 10 is offset from the storage ring 4 so that the acceleration path 3 does not intersect into the delivery system 10. This would prevent the high-energy particles from properly entering the storage ring 4. In addition, the storage ring is filled with a low-Z element, such as hydrogen or helium, at low pressure range between 10⁻⁹ Torr and 10⁻¹⁰ Torr.

The method of the present invention implements the system described above for charged particles. The charged particles can be, but is not limited to, electrons, protons, positrons, antiprotons, a helium isotope, or a carbon isotope. The method begins by producing charged particles with the particle generator 1 and accelerating the charged particles to a high-kinetic energy. This allows the charged particles to travel along the acceleration path 3 and to be injected into the input port 5. The method continues by storing a required quantity of particles within the storage ring 4 by accumulating and orbiting the charged particles along the annular storage path 7. The required quantity of particles is the number of charged particles that are needed to create the prescribed dose of ionizing radiation. The storage ring 4 allows the present invention to have the required quantity of particles to be readily available to be sent to the delivery system 10. In the preferred embodiment of the present invention, the required quantity of particles is guided along the annular storage path 7 by a magnetic field that is configured and generated by the storage ring 4. Consequently, the method proceeds by ejecting the required quantity of particles through the output port 6, if the switcher 8 is activate to redirect the required quantity of particles towards the delivery system 10. In the preferred embodiment, the switcher 8 would turn off a designated set of containment magnets for the storage ring 4, which would break the containment of the annular storage path 7 and would release the required quantity of particles towards the delivery system 10. The method continues by converting the required quantity of particles into the prescribed dose of ionizing radiation with the delivery system 10. The prescribed dose of ionizing radiation for is determined by the treatment planning system. In the preferred embodiment, the prescribed dose of ionizing radiation is either, but not limited to, X-rays or a modified particle beam. The method concludes by emitting the prescribed dose of ionizing radiation at the treatment target with the delivery system 10.

The delivery system 10 can be configured in different ways in order to create different kinds of ionizing radiation. One way is to configure the delivery system 10 with a target made of a high-Z element, which would generate X-rays as the prescribed dose of ionizing radiation once the required quantity of particles hit the target. Typically, the target is made of a metal such as tungsten, copper, or cobalt. Another way is to configure the delivery system 10 with a scattering foil and to use the required quantity of particles in a beam arrangement as the prescribed dose of ionizing radiation. The delivery system 10 would use the scattering foil to broaden the beam width of the required quantity of particles so that the prescribed dose of ionizing radiation is properly administered across the area of the treatment target. In some embodiments, the delivery system can be, but is not limited to, a compensator, a step-and-shoot multi-leaf collimator system, or an automatic compensator.

The required quantity of particles within the storage ring 4 needs to be constant so that the present invention is able to readily deliver the prescribed dose of ionizing radiation. One problem with maintaining the required quantity of particles within the storage ring 4 is that charged particles have a certain lifetime. Additional charged particles need to be continuously produced, accelerated, and injected into the storage ring 4 in order to compensate for the particle loss due to the lifetime of each charged particle within the storage ring 4. The required quantity of charged particles is a transient stable state for the number of charged particles being held within the storage ring 4. Another way to compensate for the particle loss due to the lifetime of each charged particle is utilize the non-linear dynamics of the storage ring 4 by adjusting the sextupole settings of its confinement magnets in order to improve momentum acceptance.

The charged particles within the storage ring 4 are in a bunches formation, which is where bunches of charged particles radially form around the annular storage path 7 because of Coulomb's interaction. The present invention will schedule additional charged particles to be produced, accelerated, and integrally injected into the bunches formation within the storage ring 4. This allows the present invention to maintain the proper particle density within the storage ring 4 so that the required quantity of charged particles to create the prescribed dose is readily available to be released from the storage ring 4.

Other potential alterations include converting the storage ring to a 270 degree bending tracker, as currently used in treatment head, by changing its operating parameters. The result is a conventional linear accelerator, as commonly used in radiation therapy. This conventional embodiment is capable of delivering radiation at low dose rates, with the radiation being suitable for beam modulation methods such as a multileaf collimator (MLC) and a velocity modulation transistor (VMT).

The lifetime of the charged particles, such as electrons, in the storage ring are primarily affected by Coulomb scattering among the electrons, as well as energy loss of electrons due to stopping power of gas in the storage ring. This is expressed as:

1/τ=(1/τ_{Q})+(1/τᵢₙₜᵣₐ)+(1/τₑₗₐₛ)+(1/τᵢₙₑₗₐₛ)

where 1/τ_{Q}, 1/τᵢₙₜᵣₐ, 1/τₑₗₐₛ, and 1/τᵢₙₑₗₐₛ respectively are lifetime of quantum, intra-bunch scattering, elastic scattering, and inelastic scattering.

Again to decrease scattering (of both the elastic and inelastic types), the storage ring 4 can be filled with lightweight and low pressure (in the range of 10⁻⁹ or 10⁻¹⁰ Torr) elements, such as hydrogen and helium. Resultantly, the storage ring 4 will be able to achieve a lifetime measured in hours for energies of 5 MeV or higher.

Utilizing non-linear dynamics of the ring, adjusting sextupole settings to improve momentum acceptance, can also be used to increase lifetime for charge particle in the storage ring. A stable beam intensity or current (I_{b}) is necessary for any given lifetime of particles, and is provided by continuously injecting the particles at a rate R. The relation between I_{b} and R is described as:

Ib=R×τ×(1−e^{-(1/τ)})→R×τ as t→∞

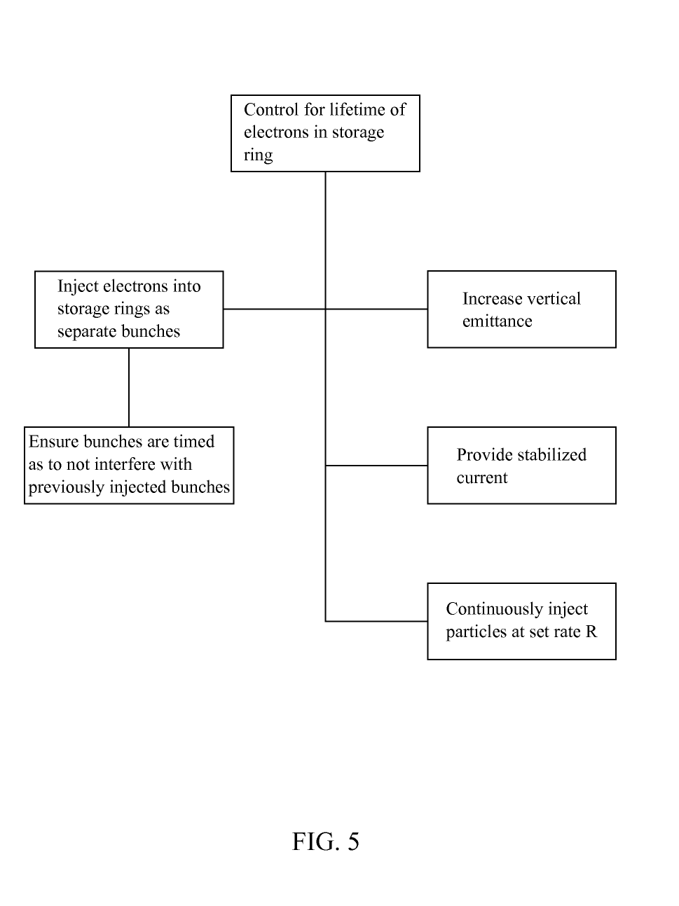

The lifetime adjustments that can be made for the present invention are outlined in FIG. 5.

To store ˜100 MU in the storage ring, the required life is τ˜⅙ minutes for injection rate around 600 MU/minute. To store 500 MU, the required life is τ˜¼ minutes for a rate around 2000 MU/minute. Resultantly, a lifetime of around 20 seconds is sufficient for most applications, while shorter lifetimes in the 5-10 second range may also be acceptable as the dose can be divided into a few short time periods.

Although the invention has been explained in relation to its preferred embodiment, it is to be understood that many other possible modifications and variations can be made without departing from the spirit and scope of the invention as hereinafter claimed.

What is claimed is:

1. A system for delivering a radiation dose with an ultra-high dose rate in radiation therapy comprises:
a particle generator;
a linear accelerator;
a storage ring;
a switcher;
a delivery system;
said storage ring comprises an input port and an output port;
said particle generator being oriented into said linear accelerator
an acceleration path for said linear accelerator being tangent to an annular storage path for said storage ring;
said acceleration path being coincident with said input port;
a release path for said storage ring being tangent to said annular storage path;
said release path being coincident with said output port;
said switcher being operatively coupled to the output port;
said release path being oriented towards said delivery system;
said acceleration path and said release path being perpendicular to each other; and
said acceleration path, said annular storage path, and said release path being coplanar to each other.

2. The system for delivering a radiation dose with an ultra-high dose rate in radiation therapy as claimed in claim 1, wherein said acceleration path and said release path are oriented at an angle of 270 degrees.

3. The system for delivering a radiation dose with an ultra-high dose rate in radiation therapy as claimed in claim 1, wherein said linear accelerator is offset from said storage ring.

4. The system for delivering a radiation dose with an ultra-high dose rate in radiation therapy as claimed in claim 1, wherein said delivery system is offset from said storage ring.

5. The system for delivering a radiation dose with an ultra-high dose rate in radiation therapy as claimed in claim 1, wherein said storage ring is filled with a low-Z element at a low pressure between 10⁻⁹ Torr and 10⁻¹⁰ Torr.

6. A method of implementing the system as claimed in claim 1, the method comprises the steps of:
producing charged particles with said particle generator;
accelerating said charged particles through said linear accelerator to a high-kinetic energy;
injecting said charged particles through said input port;
storing a required quantity of particles within said storage ring by orbiting and accumulating said charged particles along said annular storage path, wherein said required quantity of particles corresponds a prescribed dose of ionizing radiation;
ejecting said required quantity of particles through said output port,
if said switcher is activated to redirect said required quantity of particles towards said delivery system;
converting said required quantity of particles into said prescribed dose of ionizing radiation with said delivery system; and
emitting said prescribed dose of ionizing radiation at a treatment target with said delivery system.

7. The method as claimed in claim 6, wherein said charged particles contains a particle type selected from the group consisting of: electrons, protons, positrons, antiprotons, a helium isotope, and a carbon isotope.

8. The method as claimed in claim 6, wherein said delivery system is configured with a target made of a high-Z element in order to generate X-rays as said prescribed dose of ionizing radiation.

9. The method as claimed in claim 6 comprises the steps of:
wherein said required quantity of particles is used as said prescribed dose of ionizing radiation;
wherein said delivery system is configured with a scattering foil; and
broadening a beam width of said required quantity of particles with said scattering foil.

10. The method as claimed in claim 6, the method comprises the step of:
maintaining said required quantity of particles within said storage ring by continuously producing, accelerating, and injecting additional charged particles into said storage ring in order to compensate for particle loss due to a lifetime for each of said charged particles.

11. The method as claimed in claim 6, the method comprises the step of:
guiding said required quantity of particles along said annular storage path with a magnetic field, wherein said magnetic field is configured and generated by said storage ring.

12. The method as claimed in claim 6, the method comprises the steps of:
wherein said charged particles within said storage ring are in a bunches formation; and
maintaining a particle density within said storage ring for said required quantity of particles by scheduling additional charged particles to be produced, accelerated, and integrally injected into said bunches formation.
